(19) United States
(12) Patent Application Publication
(10) Pub. No.: US 2011/0046071 A1
(43) Pub. Date: Feb. 24, 2011

(54) GLP-1 Receptor Agonists And Related Active Pharmaceutical Ingredients For Treatment Of Cancer

(75) Inventors: Avraham Karasik, Ramat Hasharon (IL); Tamar Rubinek, Or Yehuda (IL); Ido Wolf, Or Yehuda (IL); Bella Kaufman, Kiryat Ono (IL)

Correspondence Address: The Law Office of Michael E. Kondoudis, 888 16th Street, N.W., Suite 800, Washington, DC, 20006, US

(73) Assignee: TEL HASHOMER MEDICAL RESEARCH INFRASTRUCTURE AND SERVICES LTD., Ramat Gan (IL)

(21) Appl. No.: 12/918,512

(22) PCT Filed: Mar. 4, 2009
(86) PCT No.: PCT/IB2009/050890

§ 371 Date: Aug. 20, 2010

Related U.S. Application Data
(60) Provisional application No. 61/033,912, filed on Mar. 5, 2008.
(60) Provisional application No. 61/033,908, filed on Mar. 5, 2008.

(51) Int. Cl. A61K 38/16; A61P 35/00

(52) U.S. Cl. 514/19.4; 514/19.3

(57) ABSTRACT

Disclosed are methods and compositions for increasing concentrations of GLP-1 receptor agonists in the body for the treatment of cancer, alone or together with other active pharmaceutical ingredients such as chemotherapeutic agents or hormone-regulating agents.

### RELATED APPLICATIONS

The present application gains priority from U.S. Provisional Patent Applications Nos. 61/033,912 and 61/033,908 both filed 5 Mar. 2008 and both which are included by reference as if fully set forth herein.

### FIELD AND BACKGROUND OF THE INVENTION

The present invention relates to the field of cancer therapy, and more particularly, in some embodiments, to the use of agents such as exendin-4 and related active pharmaceutical ingredients such as glucagon-like peptide-1 hormone, derivatives thereof, analogs thereof, mimetics thereof or salts thereof, or DPP-4 inhibitors in the treatment of cancer.

Cancer is a group of diseases in which cells grow and divide without respect to normal limits, forming a tumor, and invading and destroying adjacent tissues. Cancer cells may spread to other locations in the body, resulting in a metastatic tumor composed of cells of the same type as those of the original tumor. Cancers are treated in a number of ways including surgery (excision of a tumor), radiation therapy (directed irradiation with X-rays to destroy cancer cells) and chemotherapy (administration of APIs that are more toxic to cancer cells than to non-cancer cells). All these therapies are associated with severe side-effects, and their activity in most types of metastatic cancers is limited.

One significant cancer is breast cancer. Breast cancer is the most common cancer among women, affecting up to one in eight women in Western countries, and the second most common cause of cancer death in women in the United States. Breast cancer can metastasize to almost any other part of the body, including the lymph nodes, bones, liver, lungs, and brain. Metastatic breast cancer is incurable with a median survival period of about 2 years. Breast cancers are treated with surgery, radiation therapy, chemotherapy and, according to specific tumor characteristics, may also be treated with hormonal therapy or antibodies directed against human epidermal growth factor (HER2) protein. Treatment is also administered before (neo-adjuvant) or after (adjuvant) a surgery in which the primary tumor has been removed, in order to prevent disease recurrence. All forms of therapy, including chemotherapy, radiotherapy, hormone therapy and biological therapy may also be used.

Current chemotherapies for breast cancer are associated with severe side effects, due mainly to the inability of the chemotherapeutic agent to distinguish between normal and healthy cells, such that certain fast-growing, normal cells are also attacked. These include blood cells forming in the bone marrow and cells in the digestive tract (mouth, stomach, intestines, esophagus), reproductive system (sexual organs), and hair follicles. Some chemotherapeutic agents may affect cells of vital organs, such as the heart, kidney, bladder, lungs, and nervous system.

A chemotherapeutic agent commonly prescribed for persons suffering from breast cancer is doxorubicin, an anthracycline antibiotic that intercalates DNA. Doxorubicin is also used to treat other cancers such as leukemia, Hodgkin's lymphoma, bladder, stomach, lung, ovaries, thyroid, soft tissue sarcoma and multiple myeloma. Doxorubicin does not cross the blood-brain barrier and is therefore ineffective in treating brain metastases. Clinically-effective doses of doxorubicin used for treating cancers are accompanied by dreadful side-effects including nausea, vomiting, heart arrhythmias, neutropenia, and complete alopecia. At high cumulative doses, doxorubicin often causes cardiac side effects, such as congestive heart failure, dilated cardiomyopathy, and death due to the doxorubicin dose-dependent decline in mitochondrial oxidative phosphorylation.

Some breast cancer tumors are “hormone positive”, needing hormones (estrogen and progesterone) in order to grow. Whether a cell is hormone positive or hormone negative is determined by the presence or absence of hormone receptors. Anti-breast cancer hormone therapy prevents hormone-positive cancer cells from getting or using the natural hormones, preventing the growth thereof. The side effects of hormone therapy depend largely on the specific drug or type of treatment. Tamoxifen, which blocks estrogen, is the most common hormone treatment. Tamoxifen is associated with severe side-effects including endometrial and uterine cancer, an increase in blood triglyceride concentration, and an increased risk of thromboembolism especially during and immediately after major surgery or periods of immobility. Tamoxifen has been implicated as a cause of steatosis hepatis. A significant number of patients treated with tamoxifen experience menopause-like side effects including a reduction of libido, hot flashes, vaginal discharge, irregular menstrual periods, headaches, fatigue, nausea, vomiting, vaginal dryness or itching, irritation of the skin around the vagina, and skin rash.

It would be highly advantageous to have a treatment for cancer which is more effective and has fewer and less severe side-effects than the therapies known in the art.

### SUMMARY OF THE INVENTION

The present invention relates to the field of cancer therapy. Particularly, in some embodiments, the present invention relates to the use of agents which increase the concentration of GLP-1 receptor agonists in the body, in the treatment of cancer, as a sole active pharmaceutical ingredient or together with other active pharmaceutical ingredients such as chemotherapeutic agents.

According to one aspect of the present invention, there is provided a method of treating cancer comprising increasing the concentration of a glucagon-like peptide 1 (GLP-1) receptor agonist in the body (such as in the blood) of a subject in need thereof.

In some embodiments, increasing the concentration of GLP-1 receptor agonist in the body comprises administering to a subject in need thereof an effective amount of a composition which increases the concentration of glucagon-like peptide 1 (GLP-1) receptor agonist in the body.

According to some embodiments, the method comprises administration of a composition comprising a pharmaceutically effective amount of a glucagon-like peptide 1 (GLP-1) receptor agonist, such as a glucagon-like peptide-1 hormone, derivative thereof, analog thereof, mimetic thereof or salt thereof.

According to some embodiments, the method comprises administration of a composition comprising a pharmaceutically effective amount of a DPP-4 inhibitor.

According to another aspect of the present invention, there is provided the use of a composition for increasing the concentration of a glucagon-like peptide 1 (GLP-1) receptor agonist in the body of a subject in need thereof for the treatment of cancer.

In some embodiments, the composition comprises a pharmaceutically acceptable amount of a glucagon-like peptide 1 (GLP-1) receptor.

In some embodiments, the composition comprises a pharmaceutically acceptable amount of a DPP-4 inhibitor.

According to some embodiments of the present invention, the cancer is a cancer related to obesity and diabetes, such as, for example, cancer of the liver, esophagus, colon, ovary, endometrium, prostate or, especially, breast cancer.

According to some embodiments of the present invention, the cancer is metastatic cancer. The metastasis may be present, for example, in the lymph nodes, bones, liver, lungs, or, especially, present in the brain. In some embodiments of the present invention, the metastatic cancer is metastatic breast cancer, which may be either hormone positive or hormone negative.

According to some embodiments of the present invention, the GLP-1 receptor agonist is a GLP-1 derivative, analog, mimetic or salt thereof, or a combination of two or more GLP-1 derivatives, analogs, mimetics or salts. Preferably, the GLP-1 receptor agonist comprises exendin-4 (SEQ ID NO: 1).

According to some embodiments of the present invention, the GLP-1 receptor agonist is administered by a route selected from the group consisting of parenteral (including intravenous, intradermal, intraperitoneal, intramuscular and subcutaneous delivery), oral, nasal, buccal, sublingual, intra-tracheal, transdermal, transmucosal, and pulmonary delivery. Preferably, administration is by subcutaneous delivery.

According to some embodiments of the present invention, the DPP-4 inhibitor comprises at least one of sitagliptin, vildagliptin, saxagliptin, algoliptin, lingaliptin and Val-Pyr, derivatives thereof, analogs thereof, mimetics thereof, and salts thereof.

Generally, the dose of the GLP-1 receptor agonist administered is a dose sufficient to provide a desired beneficial effect, yet low enough so that undesirable side-effects are minimized.

According to some embodiments of the present invention, the GLP-1 receptor agonist is administered twice daily. Alternatively, the GLP-1 receptor agonist is administered in extended release form such that a clinically effective plasma level of GLP-1 receptor agonist is maintained for a period of at least 24 hours, such as, for example, 48 hours, 72 hours, 1 week, or 1 month.

According to some embodiments, the composition further comprises at least one chemotherapeutic agent as an additional active pharmaceutical ingredient, such as, for example, an alkylating agent (such as busulfan, carboplatin, carmustine, cisplatin, chloroambucil, cyclophosphamide, dacarbazine, hexamethylmelamine, ifosfamide, mechlorethamine, melphalan, oxoplatin, streptozocin, temozolomide, thiotepa, and uramustine, or combinations thereof); an antimetabolite (such as azathioprine, capecitabine, carmofur, cladribine, clofarabine, cytarabine, fludarabine, fluorouracil, gemcitabinemercaptopurine, methotrexate, premetrexed, raltitrexed, tegafur, and tioguanine, or combinations thereof); an anthracycline (such as daunorubicin, doxorubicin, epirubicin, idarubicin, mitoxantrone and valrubicin, and combinations thereof); a plant alkaloid, a topoisomerase inhibitor, a hormone receptor modulator (such as tamoxifen or faslodex), a hormone level modulator (such as letrozole, anastrazole or aromasin); herceptin, lapatinib, bevacizumab, cetuximab panitumumab, erlotinib, and sunitinib, or combinations thereof.

According to some embodiments, the chemotherapeutic agent comprises doxorubicin.

According to some embodiments, the chemotherapeutic agent comprises at least one of herceptin, lapatinib, bevacizumab, cetuximab panitumumab, erlotinib, and sunitinib.

According to some embodiments, the GLP-1 receptor agonist and the chemotherapeutic agent are administered in a single dosage form. Alternatively, the GLP-1 receptor agonist and the chemotherapeutic agent may be administered sequentially in separate dosage forms.

According to yet another aspect of the present invention, there is provided a method of treating cancer comprising administering to a subject in need thereof a pharmaceutically effective amount of a composition comprising exendin-4, a derivative thereof, an analog thereof, a mimetic thereof, or salts thereof.

According to still another aspect of the present invention, there is provided the use of a composition comprising a pharmaceutically effective amount of exendin-4, a derivative thereof, an analog thereof, a mimetic thereof, or salts thereof, for the treatment of cancer.

According to some embodiments, the analog, derivative, mimetic, or salt of exendin-4 is a GLP-1 receptor agonist.

According to some embodiments, the cancer is a cancer related to obesity and diabetes, such as, for example, cancer of the liver, esophagus, colon, ovary, endometrium, prostate or, especially, breast cancer.

According to some embodiments of the present invention, the cancer is metastatic cancer. The metastasis may be present, for example, in the lymph nodes, bones, liver, lungs or, especially present in the brain. In some embodiments of the present invention the metastatic cancer is metastatic breast cancer, which may be either hormone positive or hormone negative.

According to some embodiments, the exendin-4 or derivative or analog or mimetic or salt may be administered by parenteral (such as intravenous, intradermal, intraperitoneal, intramuscular or subcutaneous delivery), oral, nasal, buccal, sublingual, intra-tracheal, transdermal, transmucosal, or pulmonary delivery routes. Preferably, the route comprises subcutaneous delivery.

Generally, the dose of the exendin-4, or derivative, or analog, or mimetic, or salt administered is a dose sufficient to provide a desired beneficial effect, yet low enough so that undesirable side-effects are minimized.

According to some embodiments, the exendin-4, or derivative, or analog, or mimetic, or salt, is administered twice daily. Alternatively, exendin-4 may be administered in extended release form such that a clinically effective plasma level of exendin-4 is maintained for a period of at least 24 hours, such as, for example, 48 hours, 72 hours, 1 week or 1 month.

According to some embodiments, the composition further comprises at least one chemotherapeutic agent as an additional active pharmaceutical ingredient in addition to exendin-4, or derivative, or analog, or mimetic, or salt thereof. Suitable chemotherapeutic agents include, for example, an alkylating agent (such as busulfan, carboplatin, carmustine, cisplatin, chloroambucil, cyclophosphamide, dacarbazine, hexamethylmelamine, ifosfamide, mechlorethamine, melphalan, oxoplatin, streptozocin, temozolomide, thiotepa, and uramustine, or combinations thereof); an antimetabolite (such as azathioprine, capecitabine, carmofur, cladribine, clofarabine, cytarabine, fludarabine, fluorouracil, gemcitabinemercaptopurine, methotrexate, premetrexed, raltitrexed, tegafur, and tioguanine, or combinations thereof); an anthracycline (such as daunorubicin, doxorubicin, epirubicin, idarubicin, mitoxantrone and valrubicin, and combinations thereof); a plant alkaloid, a topoisomerase inhibitor, a hormone receptor modulator (such as tamoxifen or faslodex), a hormone level modulator (such as letrozole, anastrazole or aromasin); herceptin, lapatinib, bevacizumab, cetuximab panitumumab, erlotinib, and sunitinib, or combinations thereof.

According to some embodiments, the exendin-4, or derivative, or analog, or mimetic, or salt thereof, and the chemotherapeutic agent, are administered in a single dosage form. Alternatively, the exendin-4, or derivative, or analog, or mimetic, or salt thereof, and the chemotherapeutic agent, may be administered sequentially or simultaneously in separate dosage forms.

According to yet another aspect of the present invention, there is provided a method of treating breast cancer comprising administering to a subject in need thereof an effective amount of a composition comprising a pharmaceutically effective amount of exendin-4, a derivative thereof, an analog thereof, a mimetic thereof, or salts thereof.

According to yet another aspect of the present invention, there is provided a composition comprising a pharmaceutically acceptable amount of exendin-4, a derivative thereof, an analog thereof, a mimetic thereof, or salts thereof, for the treatment of breast cancer.

According to some embodiments, the analog, mimetic, derivative or salt of exendin-4 is a GLP-1 receptor agonist.

According to some embodiments, the exendin-4, or derivative, or analog, or mimetic, or salt, is administered by a route selected from the group consisting of parenteral (including intravenous, intradermal, intraperitoneal, intramuscular and subcutaneous delivery), oral, nasal, buccal, sublingual, intra-tracheal, transdermal, transmucosal, and pulmonary delivery. Preferably, administration is by subcutaneous delivery.

According to yet another aspect of the present invention there is provided a composition comprising pharmaceutically effective amounts of a GLP-1 receptor agonist and at least one chemotherapeutic agent as an additional active pharmaceutical ingredient, and a pharmaceutically effective carrier.

According to some embodiments, the GLP-1 receptor agonist is a GLP-1 analog, mimetic, derivative or salt. Preferably, the derivative comprises exendin-4.

Typical chemotherapeutic agents include, for example, an alkylating agent (such as busulfan, carboplatin, carmustine, cisplatin, chloroambucil, cyclophosphamide, dacarbazine, hexamethylmelamine, ifosfamide, mechlorethamine, melphalan, oxoplatin, streptozocin, temozolomide, thiotepa, and uramustine, or combinations thereof); an antimetabolite (such as azathioprine, capecitabine, carmofur, cladribine, clofarabine, cytarabine, fludarabine, fluorouracil, gemcitabinemercaptopurine, methotrexate, premetrexed, raltitrexed, tegafur, and tioguanine, or combinations thereof); an anthracycline (such as daunorubicin, doxorubicin, epirubicin, idarubicin, mitoxantrone and valrubicin, and combinations thereof); a plant alkaloid, a topoisomerase inhibitor, a hormone receptor modulator (such as tamoxifen or faslodex), a hormone level modulator (such as letrozole, anastrazole or aromasin); herceptin, lapatinib, bevacizumab, cetuximab panitumumab, erlotinib, and sunitinib, or combinations thereof.

According to some embodiments, the composition comprises a pharmaceutically effective amount of exendin-4 (as the GLP-1 receptor agonist) and of doxorubicin (as the chemotherapeutic agent), and a pharmaceutically effective carrier.

According to some embodiments, there is provided a composition comprising a combination of at least two active pharmaceutical ingredients, wherein a first active pharmaceutical ingredient is a GLP-1 receptor agonist and a second active pharmaceutical ingredient is a chemotherapeutic agent, wherein the amount of GLP-1 receptor agonist alone and the amount of chemotherapeutic agent alone is insufficient to achieve the therapeutic effect achieved by the administration of the combination of two or more active pharmaceutical ingredients.

According to some embodiments, the GLP-1 receptor agonist comprises exendin-4.

According to some embodiments, the chemotherapeutic agent comprises doxorubicin.

The amount of exendin-4 that is approved for administration for the treatment of diabetes is currently about 10 microgram/day.

In some embodiments of any of the aspects of the present invention wherein a composition comprises exendin-4, the dose of exendin-4 administered is similar to that approved for administration for the treatment of diabetes. According to some embodiments wherein a composition comprises exendin-4, the dose of exendin-4 administered is from about 0.2 microgram/day to about 20 microgram/day, and in some embodiments, from about 0.2 microgram/day to about 10 microgram/day.

Since at a 10 microgram/day dose of exendin-4, some subjects experience nausea, in some embodiments the dose of exendin-4 is less than 10 microgram/day. For example, in some embodiments, exendin-4 is administered in a dose of from about 0.2 microgram/day to about 5 microgram/day, and in some embodiments from about 0.2 microgram/day to about 1 microgram/day.

In some embodiments, wherein exendin-4 is administered together with some other active pharmaceutical ingredient, whether simultaneously or serially, the co-administration allows reduction of the dose of exendin-4 administered.

In some embodiments, wherein the use for which the composition is administered allows, the dose of exendin-4 administered is lower than that which would normally be required to produce a therapeutic effect.

In some embodiments, where a composition comprises a glucagon-like peptide 1 (GLP-1) receptor agonist other than exendin-4 as an active pharmaceutical ingredient, the dose of active pharmaceutical ingredient administered is a dose that is pharmaceutically equivalent to the dose of exendin-4, as discussed above.

According to another aspect of the present invention, there is provided a method of treating cancer comprising administering to a subject in need thereof a pharmaceutically effective amount of a composition comprising a DPP-4 inhibitor.

According to yet another aspect of the present invention, there is provided the use of a composition comprising a pharmaceutically effective amount of a DPP-4 inhibitor for the treatment of cancer

In some embodiment, the DPP-4 inhibitor comprises sitigalipin, which is administered in a dose of from about 0.1 microgram to about 200 milligram/day.

In some embodiments, the dose is about 100 milligram/day.

In some embodiments, the sitagliptin is administered once daily.

In some embodiments, the DPP-4 inhibitor is administered in extended release form such that a clinically effective plasma level of the exendin-4 the analog, the mimetic, the derivative, or the salt thereof is maintained for a period of at least 24 hours.

According to yet another aspect of the present invention, there is provided a method of treating breast cancer comprising administering to a subject in need thereof an effective amount of a composition comprising a pharmaceutically effective amount of a DPP-4 inhibitor.

According to yet another aspect of the present invention, there is provided the use of a composition comprising a pharmaceutically acceptable amount of a DPP-4 inhibitor for the treatment of breast cancer.

According to yet another aspect of the present invention, there is provided a composition comprising pharmaceutically effective amounts of a DPP-4 inhibitor and doxorubicin, and a pharmaceutically effective carrier.

According to yet another aspect of the present invention, there is provided a composition comprising a combination of at least two active pharmaceutical ingredients, wherein a first the active pharmaceutical ingredient is at least one DPP-4 inhibitor and a second the active pharmaceutical ingredient is at least one chemotherapeutic agent, wherein the amount of the at least one DPP-4 inhibitor alone and the amount of the at least one chemotherapeutic agent alone is insufficient to achieve the therapeutic effect achieved by the administration of the combination of two or more of the agents.

In some embodiments, the DPP-4 inhibitor comprises sitagliptin.

In some embodiments, the chemotherapeutic agent comprises doxorubicin.

Unless otherwise defined, all technical and scientific terms used herein have the same meaning as commonly understood by one of ordinary skill in the art to which this invention pertains. In case of conflict, the patent specification, including definitions, will control.

As used herein, a “GLP-1 receptor agonist” is taken to be any compound, including peptides and non-peptide compounds, which fully or partially binds to the human GLP-1 receptor and triggers a response.

As used herein, a DPP-4 inhibitor is taken to be a compound, including peptides and non-peptide compounds, which fully or partially inhibits the enzymatic function of DPP-4 of degrading glucagon-like peptide-1 (GLP-1).

As used herein, by breast cancer is intended both female breast cancer and male breast cancer.

As used herein, the term “treating” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “pharmaceutically effective amount” denotes that dose of an active pharmaceutical ingredient or a composition comprising the active pharmaceutical ingredient that will provide the therapeutic effect for which the active pharmaceutical ingredient is indicated.

As used herein, the term “pharmaceutically acceptable” means approved by a regulatory agency of the Federal or a state government or listed in the U.S. Pharmacopeia or other generally recognized pharmacopeia for use in animals, and more particularly in humans. Herein, the phrase “pharmaceutically acceptable carrier” refers to an approved carrier or a diluent that does not cause significant irritation to an organism and does not abrogate the biological activity and properties of the administered conjugate.

As used herein, the term “carrier” refers to a diluent, adjuvant, excipient, or vehicle with which the therapeutic is administered.

Herein the term “excipient” refers to an inert substance added to a pharmaceutical composition to further facilitate processes and administration of the active pharmaceutical ingredients.

As used herein, the indefinite articles “a” and “an” mean “at least one” or “one or more” unless the context clearly dictates otherwise. For example, when a composition is described as comprising an active pharmaceutical ingredient, it is understood that the intention is that the composition comprises at least one active pharmaceutical ingredient unless otherwise specified.

### BRIEF DESCRIPTION OF THE FIGURES

Some embodiments of the invention are herein described, by way of example only, with reference to the accompanying figures. The description, together with the figures, makes apparent how embodiments of the invention may be practiced to those skilled in the art. It is stressed that the particulars shown in the figures are by way of example and for purposes of illustrative discussion of embodiments of the invention.

In the figures:

FIGS. 1A-1D are bar charts showing the dose-dependent effect of exendin-4 on the viability of MCF-7 (1A), MDA-MB-231 (1B), human primary liver cells (1C), and human embryonic kidney cells (1D);

FIG. 2 is a bar chart summarizing the results of FIGS. 1A-1C;

FIG. 3 shows the effect of exendin-4 on cell growth;

FIG. 4 shows the effect of exendin-4 on expression of p53 and p21;

FIG. 5 is a bar chart showing the increase of cAMP with time following exendin-4 treatment;

FIG. 6A is a bar chart showing the effect of different concentrations of exendin-4 on cAMP levels; and FIG. 6B is a bar chart showing the effect on cAMP levels of exendin-4, GLP-1, and exendin (9-3), alone or in combination;

FIG. 7 is a bar chart showing the effects of exendin-4 and forskolin, alone or in combination, on viability of MCF-7 cells;

FIG. 8 is a bar chart showing the effects of exendin-4, GLP-1, forskolin, exendin (9-39) and dda, alone or in combination, on cAMP production in MCF-7 cells;

FIG. 9 is a bar chart showing the effects on viability of MCF-7 breast cancer cells of doxorubicin and exendin-4, alone and in combination;

FIG. 10 shows the effect of exendin-4 on p38 and CREB activation;

FIG. 11 shows the effect of exendin-4 and exendin (9-3) on AMPK and CREB activation; and

FIG. 12 is a graph showing in vivo inhibition of breast tumors by exendin-4.

### DESCRIPTION OF SOME EMBODIMENTS

The present invention relates to methods of treatment of cancer, the uses of various active pharmaceutical ingredients for the treatment of cancer and to compositions for use in the treatment of cancer.

Two major factors affecting breast cancer incidence and outcome are obesity and type 2 diabetes mellitus. Several factors may associate obesity and diabetes with elevated breast cancer risk, and include activated insulin and insulin-like growth factor 1 (IGF-1) pathways, altered regulation of endogenous sex-hormones and altered levels of adipocytokines.

The incretin system is a network of hormones, including glucagon-like peptide 1, which are secreted from the gastrointestinal tract in response to food ingestion and enhance glucose stimulated insulin secretion.

Glucagon-like peptide 1 (GLP-1) is an endogenous peptide-hormone, comprising a sequence of 31 amino acids, which is secreted from endocrine L cells, located in the distal ileum and colon, in response to food intake. GLP-1 stimulates insulin secretion and sensitivity, and inhibits glucagon secretion. It increases pdx-1 gene transcription and enhances the binding of Pdx-1 to the insulin gene promoter.

In addition, GLP-1 promotes pancreatic β-cell proliferation. GLP-1 also appears to be a physiological regulator of appetite and food intake, which promotes satiety and inhibits gastric emptying. Sustained activation of the GLP-1 receptor is associated with weight loss.

These actions are mediated mainly through the GLP-1 receptor (GLP-1R), a seven transmembrane G-protein coupled receptor, expressed on pancreatic β cells and in many other cell types, including brain, heart and muscle cells. Due to these actions, GLP-1 and GLP-1R were considered potential targets for use in diabetes mellitus type 2 therapy.

Exendin-4 (SEQ ID No:1. CAS 141732-76-5, a 39 amino acid residue polypeptide having the sequence: HGEGTFTSDLSKQMEEEAVRLFIEWLKNGGP SSGAPPPS), a GLP-1 receptor agonist, has been approved by the US FDA for the treatment of type 2 diabetes. In a separate study, it was reported that while exendin-4 increases pancreatic β-cell proliferation, and reduces apoptosis of such cells, it has no effect on cell proliferation or apoptosis of pancreatic cancer cell lines (Diabetes 55:1369 (2006)).

As described in detail in the Examples section below, it has been surprisingly found by the present inventors that, in contrast to what is known in the art, at least some GLP-1 receptor agonists are useful in affecting cancer cells and therefore in the treatment of cancer.

If GLP-1 receptor agonists are useful in affecting cancer cells then one way of treating cancer is by increasing the amount of GLP-1 receptor agonists in the body of the subject. This may be achieved by administering exogenous GLP-1 receptor agonists, or by increasing the concentration in the body, such as in the blood, of endogenous GLP-1 receptor agonists.

Thus, in some embodiments the present invention provides a method of treatment of cancer comprising administering to a subject in need thereof a composition which increases the concentration of GLP-1 receptor agonist in the body.

In some embodiments, increasing the concentration of GLP-1 receptor agonist comprises administering an effective amount of a glucagon-like peptide 1 receptor agonist.

The concentration of endogenous GLP-1 receptor agonists in the body may be increased by inhibiting enzymes which degrade endogenous GLP-1 receptor agonists.

DPP-4 (dipeptidyl peptidase 4, also known as CD 26) is a membrane-associated peptidase of 766 amino acids that is widely distributed in numerous tissues. DPP-4 also exists as a soluble circulating form in plasma and significant DPP-4-like activity is detectable in plasma from humans and rodents. DPP-4 exerts its biological effects via two distinct mechanisms of action. First, as a membrane spanning protein, DPP-4 binds adenosine deaminase and when activated, conveys intracellular signals independent of its enzymatic function via dimerization and activation of intracellular signaling pathways. Second, DPP-4 has an enzymatic function. The enzymatic activity of DPP-4 is exhibited both by the membrane-spanning form of the molecule, and by the slightly smaller circulating soluble form. DPP-4 prefers substrates with an amino-terminal proline or alanine at position 2, but may also cleave substrates with non-preferred amino acids at position 2. The structure of GIP, GLP-1 and GLP-2 reveals a highly conserved alanine at position 2. Observations from a number of laboratories delineated the importance of DPP-4-mediated inactivation of GLP-1 as a key determinant of GLP-1 bioactivity. Studies have confirmed that DPP-4-mediated inactivation of these peptides is a critical control mechanism for regulating the biological activity of both GIP and GLP-1 in vivo.

Therefore, according to some embodiments of the present invention, a DPP-4 inhibitor is administered to a subject in need thereof. Without wishing to be held to any one theory, the DPP-4 inhibitor inhibits the DPP-4-mediated cleavage of endogenous active GLP-1 which then is available to exercise an anti-cancer effect acts as a GLP-1 receptor agonist. Thus it is believed that in some embodiments of the present invention, enzymes (such as DPP-4) that degrade endogenous GLP-1 receptor agonists are inhibited, increasing the anticancer efficacy of the endogenous GLP-1 receptor agonists to a clinically useful extent. An increase of concentration of GLP-1 subsequent to administration of a DPP-4 inhibitor has been demonstrated, see for example, Herman G A et al in J Clin Pharmacol 2006, 46, 876-886.

DeFronzo R A in “Treatment of Type 2 Diabetes: Role of GLP-1 Analogues and DPP-4 Inhibitors” increase in serum GLP-1 levels in diabetic humans following oral administration of Vildagliptin

Thus, in some embodiments the present invention provides a method of treatment of cancer comprising administering to a subject in need thereof an effective amount of a DPP-4 inhibitor.

In some embodiments, the present invention further provides the use of a composition which increases the concentration of GLP-1 receptor agonist in the body, e.g., the blood.

In some embodiments, the composition comprises a pharmaceutically acceptable amount of a glucagon-like peptide 1 (GLP-1) receptor agonist for the treatment of cancer. In some embodiments, the composition comprises a pharmaceutically acceptable amount of a DPP-4 inhibitor for the treatment of cancer.

According to some embodiments of the present invention, the cancer may be any cancer which is associated with diabetes or obesity, such as, for example, cancer of the liver, esophagus, colon, ovary, endometrium, prostate or, especially, breast. The breast cancer may be either hormone-positive or hormone-negative breast cancer.

According to some embodiments of the present invention, the cancer may be metastatic cancer. The metastatic cancer may be present, for example, in lymph nodes, bones, liver, lungs, and brain.

It is known that some GLP-1 receptor agonists, such as exendin-4, easily pass the blood brain barrier, which is usually impervious to anti-cancer agents. Therefore, in some embodiments, the teachings of the present invention may be applied to treating metastatic cancer in the brain.

In some embodiments, a GLP-1 receptor agonist is used to treat metastatic cancer in the brain.

In some embodiments, a DPP-4 inhibitor is used to treat metastatic cancer in the brain. The DPP-inhibitor of the present invention effectively crosses the blood-brain barrier to exercise the desired anti-cancer effect in the brain. Since it is known that GLP-1 is able to cross the blood-brain barrier, the teachings of the present invention lead to an increase in the amount of GLP-1 in the body, which crosses the blood-brain barrier to exercise the desired anti-cancer effect in the brain.

In some embodiments, the metastatic cancer comprises metastases of cancers related to obesity and diabetes, such as liver, esophagus, colon, ovary, endometrium, prostate and especially breast cancers

The GLP-1 receptor agonist of the present invention may be any GLP-1 analog or derivative, such as those described in detail in U.S. Pat. No. 5,424,286 and the PCT patent applications published as WO 98/08871, WO 99/43706, and WO 00/09666, which are all incorporated by reference as if enclosed herein in their entirety.

Particular examples include GLP-1, such as human GLP-1 and exendin-4, and analogs or derivatives thereof. In an embodiment, the GLP-1 compound is a fragment of human GLP-1 (1-37) or exendin-4 (1-39), such as human GLP-1 (7-37) wherein the amino acid residues in positions 1-6 of human GLP-1 (1-37) have been deleted, and human GLP-1 (7-36) where the amino acid residues in position 1-6 and 37 of human GLP-1 (1-37) have been deleted, exendin-4 (1-38) where amino acid residue 39 has been deleted from exendin-1 (1-39) and exendin-4 (1-31), where amino acid residue 32-39 have been deleted from exendin-4 (1-39). In an embodiment, the GLP-1 compound is an analog of human GLP-1 (1-37) or exendin-4 (1-39), such as Met8-GLP-1 (7-37) wherein the alanine in position 8 has been replaced by methionine and the amino acid residues in position 1 to 6 have been deleted relative to human GLP-1 (1-37); Arg34-GLP-1 (7-37) wherein valine in position 34 has been replaced with arginine and the amino acid residues in position 1 to 6 have been deleted relative to human GLP-1 (1-37); and Ser2Asp3-exendin-4 (1-39) wherein the amino acid residues in position 2 and 3 have been replaced with serine and aspartic acid relative to exendin-4(1-39), respectively (this particular analog also being known in the art as exendin-3). In an embodiment, the GLP-1 compounds is a derivative of human GLP-1 (1-37) or exendin-4(1-39), such as GLP-1 (7-36)-amide, Arg34 and Lys26(Nε-(γ-Glu(Nα-hexadecanoyl)))-GLP-1 (7-37). The GLP-1 receptor agonists of the present invention may be prepared using any method known in the art, such as, for example recombinant or standard solid-phase peptide synthesis techniques and preferably an automated or semiautomated peptide synthesizer. An example of the preparation of exendin-3 and exendin-4 is described in the US patent application published as US 2006/0183677. The preparation of additional exendin agonist peptide analogs is described in, for example, the PCT patent application published as WO 00/41546.

GLP-1 itself has a short half life (less than about 2 minutes) in the circulation, and is rapidly inactivated by the ubiquitous proteolytic enzyme dipeptidyl peptidase-4 (DPP-4). A short half life is inconvenient in cases where it is desired to maintain a high blood level of an active pharmaceutical ingredient over a prolonged period of time since repeated administrations will then be necessary.

Analogs and derivatives of GLP-1 and GLP-1R having a relatively long half-life have been investigated for their potential benefits in the treatment of diabetes (Klonoff D C et al. Exenatide effects on diabetes, obesity, cardiovascular risk factors and hepatic biomarkers in patients with type 2 diabetes treated for at least 3 years. Curr Med Res Opin 2008 January; 24(1): 275-86).

It was therefore considered by the present inventors, that GLP-1 receptor agonists having an extended half-life would be particularly useful in the method of the present invention. Preferably, the GLP-1 receptor agonist of the present invention has a half-life in the circulation of at least 2 hours.

One such GLP-1 receptor agonist is exendin-4, which is isolated from the venom of Heloderma suspectum (Gila monster). Exendin-4 is a potent GLP-1 receptor agonist, having a half life in the circulation of about 2-4 hours. Exendin-4 has shown efficacy in the treatment of diabetic patients and its administration is associated with improved glycemic control, weight loss and reduced insulin resistance.

Exendin-4 increases insulin sensitivity and reduces weight, two factors known to be associated with reduced incidence of breast cancer. It was therefore hypothesized by the present inventors that exendin-4 may also antagonize insulin activities in breast cancer cells and thus inhibit their growth,

A synthetic version of exendin-4 (exenatide, CAS 141732-76-5, SEQ ID NO: 1) has been approved by the United States FDA for the treatment of type 2 diabetes mellitus and is marketed as Byetta® (Amylin Pharmaceuticals, San Diego, Calif., USA and Eli Lilly and Company, Indianapolis, Ind., USA) which is also available in extended-release formulation Exenatide LAR (long-acting-release. Another GLP-1 receptor agonist undergoing the process of approval for the treatment of diabetes includes liraglutide (CAS 204656-20-2, to be marketed under the brand-name Victoza® by Novo Nordisk A/S, Bagsvaerd, Denmark) having the IUPAC name L-histidyl- L-alanyl- L-α-glutamylglycyl- L-threonyl- L-phenylalanyl- L-threonyl- L-seryl- L-α-aspartyl- L-valyl- L-seryl- L-seryl- L-tyrosyl- L-leucyl- L-α-glutamylglycyl- L-glutaminyl- L-alanyl- L-alanyl-N⁶-[N-(1-oxohexadecyl)-L-γ-glutamyl]- L-lysyl- L-α-glutamyl- L-phenylalanyl- L-isoleucyl- L-alanyl- L-tryptophyl- L -leucyl- L-valyl- Larginylglycyl- L-arginyl-glycine.

While exendin-4 increases pancreatic β-cell proliferation, and reduces apoptosis of such cells, it has no effect on cell proliferation or apoptosis of pancreatic cancer cell lines (Diabetes 55: 1369 (2006)).

A DPP-4 inhibitor used in implementing the teachings of the present invention may be any effective DPP-4 inhibitor known in the art, derivatives thereof, analogs thereof, mimetics thereof, and salts thereof. In some embodiments a single DPP-4 inhibitor is used in implementing the teachings of the present invention. In some embodiments a combination of two or more DPP-4 inhibitors are used in implementing the teachings of the present invention.

Known DPP-4 inhibitors that in some embodiments may be useful in implementing the teachings of the present invention include, but are not limited to, sitagliptin, vildagliptin, saxagliptin, algoliptin, lingaliptin and Val-Pyr.

Sitagliptin ((2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine, CAS 790712-60-6, available as Januvia™ from Merck & Co., Inc., Whitehouse Station, N.J., USA) is a DPP-4 inhibitor that has been approved for the treatment of type 2 diabetes in the United States. Sitagliptin has been approved for use either as monotherapy, or as combination therapy with either metformin or a thiazolidinedione (e.g., rosiglitazone or pioglitazone). The usual dose is 100 mg daily. Subjects with renal impairment may require a reduction in dose to either 50 mg or 25 mg. Sitagliptin is cleared via the kidney, exhibits a half life of 8-12 hours, and a single dose of 100 mg produces long lasting DPP-4 inhibition over a 24 hr period. The majority (74%) of sitagliptin is eliminated without being metabolized in human subjects, via the kidney, with a small amount detected in the GI tract.

Vildagliptin ((2S)-1-[N-(3-hydroxy-1-adamantyl)glycyl] pyrrolidine-2-carbonitrile, CAS 274901-16-5, expected to be available as Galvus™ from Novartis International AG, Basel, Switzerland) is expected to be approved for treatment of type-2 diabetes.

Saxagliptin ((1S,3S,5S)-2-[(2S)-2-amino-2-(3-hydroxy-1-adamantyl(acetyl]-2-azabicyclo[3.1.0]hexane-3-carbonitrile, CAS 361442-04-8), expected to be available from Bristol-Myers Squibb, New York, N.Y., USA) is expected to be approved for the treatment of type-2 diabetes.

Algoliptin (2-({6-[(3R)-3-aminopiperidin-1-yl]-3-methyl-2,4-dioxo-3,4-dihydropyrimidin-1(2H)-yl}methyl)benzonitrile, CAS 850649-62-6) is being developed by Takeda Pharmaceutical Company (Osaka, Japan) is expected to be approved for the treatment of type-2 diabetes.

Linagliptin (8-[(3R)-3-aminopiperidin-1-yl]-7-(but-2-yn-1-yl)-3-methyl-1-[(4-methylquinazolin-2-methyl]-3,7-dihydro-1H-purine-2,6-dione, CAS 668270-12-0) is being developed by Boehringer Ingelheim (Ingelheim, Germany) is being developed for the treatment of type-2 diabetes.

The GLP-1 receptor agonist compounds or DPP-4 inhibitors can be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

As described in detail in the Examples section below, short-term effects of exendin-4 on the proliferation of breast cancer cells were tested using MTT assays and long-term effects were examined using colony formation assays. The viability of both hormone-positive (MCF-7) and hormone-negative (MDA-MB-231) breast cancer cell lines was shown to be reduced by 50% by exendin-4, while proliferation of normal mammary cell line HB2, human embryonic kidney HEK-293, and human primary liver cells was not affected.

Similarly, clonogenic assays showed that exendin-4 inhibited proliferation of both hormone-positive and hormone-negative breast cancer cell lines, but elevated proliferation of human primary liver cells. Hence, herein exendin-4 is shown, for the first time, to be effective as an anti-cancer agent.

In vivo, exendin-4 delivery inhibited tumor growth of MDA-MB-231 cells injected to the flanks of athymic mice.

Exendin-4 treatment was also found to induce elevation of the tumor suppressors p53 and p21 in breast cancer cells, but to cause down-regulation in liver primary cultures, further confirming the role of exendin-4 as an anti-cancer agent.

Furthermore, exendin-4 treatment increased apoptosis of breast cancer cells, as evidenced by increased annexin-v staining and poly ADP ribose polymerase (PARP) cleavage, whereas apoptosis of primary liver cells was decreased.

Downstream signaling pathways that modulate GLP-1 activity were evaluated. The classic GLP-1 receptor was not detected in breast cancer cells, but treatment with either exendin-4 or GLP-1 elevated cyclic 3′,5′,7′-adenosine monophosphate phosphodiesterase (cAMP) levels, suggesting the existence of a non-classical GLP-1 receptor in these cells. Studies reported below revealed stimulation of cAMP production in MCF-7 and MDA-MB-231 breast cancer cells, as well as increased AMP kinase activity, in MCF-7 cells following exendin-4 treatment, suggesting that a G-protein-coupled receptor is activated in these cells. A peak in cAMP level is seen after 5 minutes, which is consistent with activation of a G-protein-coupled receptor.

GLP-1 and exendin-4 treatment induced p38 mitogen-activated protein kinase (MAPK) and cAMP response element binding protein (CREB) phosphorylation. Neither GLP-1 nor exendin-4 affected the extracellular signal-regulated kinase (ERK)1/2 or AKT pathways.

Taken together, these results show that exendin-4, which has already been approved by the US FDA for treatment of type 2 diabetes, is an effective and selective inhibitor of breast cancer cell proliferation. If GLP-1 receptor agonists are useful in affecting cancer cells then one way of treating cancer is by increasing the amount of GLP-1 receptor agonists in the body of the subject. As discussed above, GLP-1 is an endogenous GLP-1 receptor agonist that is enzymatically inactivated by DPP-4. By inhibiting the deactivation of GLP-1 by DPP-4, more GLP-1 may be available to exercise the unexpectedly discovered anti-cancer effect of GLP-1 receptor agonists.

Therefore, according to some embodiments of the present invention, there is provided a method of treating cancer comprising administering to a subject in need thereof a pharmaceutically effective amount of a composition comprising exendin-4, or an analog, mimetic, derivative or salt thereof.

According to some embodiments of the present invention, there is provided a method of treating cancer comprising administering to a subject in need thereof a pharmaceutically effective amount of a composition comprising a DPP-4 inhibitor.

Also, according to some embodiments of the present invention, there is provided a method of treating breast cancer comprising administering to a subject in need thereof a pharmaceutically effective amount of a composition comprising exendin-4.

Further, according to some embodiments of the present invention, there is provided a method of treating breast cancer comprising administering to a subject in need thereof a pharmaceutically effective amount of a composition comprising a DPP-4 inhibitor.

The GLP-1 receptor agonist or the DPP-4 inhibitor of the present invention may be administered by parenteral (including intravenous, intradermal, intraperitoneal, intramuscular and subcutaneous) routes. Alternatively, the GLP-1 receptor agonist or DPP-4 inhibitor may be administered by alternative delivery routes, including oral, nasal, buccal, sublingual, intra-tracheal, transdermal, transmucosal, and pulmonary. The GLP-1 receptor agonist or DPP-4 inhibitor may be administered by continuous release or delivery, using, for example, an infusion pump, continuous infusion, controlled release formulations utilizing polymer, oil or water insoluble matrices.

Carriers or excipients known in the art can also be used to facilitate administration of the GLP-1 receptor agonists or DPP-4 inhibitors of the present invention. Examples of carriers and excipients include calcium carbonate, calcium phosphate, various sugars such as lactose, or types of starch, cellulose derivatives, gelatin, vegetable oils, polyethylene glycols and physiologically compatible solvents.

According to some embodiments, the GLP-1 receptor agonist or DPP-4 inhibitor may be administered in extended release form, which is capable of releasing the agonist over a predetermined release period, such that a clinically effective plasma level of the agonist is maintained for at least 24 hours, such as at least 48 hours, at least 72 hours, at least one week, or at least one month. For example, an injectable, extended release formulation may be prepared, similar to analogous to the extended-release formulation of exenatide available from Eli Lilly and company, or by encapsulating the GLP-1 receptor agonist into injectable, biodegradable polymer microspheres or liposomes. Optionally, polyethylene glycol (PEG)-lipids may be incorporated into the liposomes in order to further increase the time for which the liposome remains in the blood. Alternatively, an injectable, extended release formulation may comprise a carrier containing a biocompatible hydrophobic vehicle such as an oil and an effective amount of polyglycerol ester.

According to some embodiments of the present invention, the GLP-1 receptor agonist, such as exendin-4, or the DPP-4 inhibitor, may be administered in combination with one or more chemotherapeutic agents, including, for example, alkylating agents, antimetabolites, anthracyclines, plant alkaloids, topoisomerase inhibitors, hormone receptor modulators, hormone level modulators, and other antitumour agents.

Examples of suitable alkylating agents include, without limitation, busulfan, carboplatin, carmustine, cisplatin, chloroambucil, cyclophosphamide, dacarbazine, hexamethylmelamine, ifosfamide, mechlorethamine, melphalan, oxoplatin, streptozocin, temozolomide, thiotepa, and uramustine.

Non-limiting examples of suitable antimetabolites include azathioprine, capecitabine, carmofur, cladribine, clofarabine, cytarabine, fludarabine, fluorouracil, gemcitabine, mercaptopurine, methotrexate, premetrexed, raltitrexed, tegafur, and tioguanine.

Suitable anthracyclines include, for example, daunorubicin, doxorubicin, epirubicin, idarubicin, mitoxantrone and valrubicin.

Examples of suitable plant alkaloids include docetaxel, paclitaxel, vinblastine, vincristine, vindesine, and vinorelbine.

Examples of suitable topoisomerase inhibitors include amsacrine, etoposide, etoposide phosphate, irinotecan, teniposide, and topotecan.

Examples of suitable hormone receptor modulators include tamoxifen; and estrogen antagonists, such as faslodex.

Examples of suitable hormone level modulators include aromatose inhibitors, such as letrozole, anastrazole and aromasin.

Examples of other antitumor agents include dactinomycin, and other chemotherapeutic agents for treatment of obesity-related cancers, such as trastuzumab (herceptin), lapatinib, bevacizumab (avastin), cetuximab (erbitux), panitumumab, erlotinib, and sunitinib.

Preferably, the chemotherapeutic agent comprises an anthracycline. More preferably, the anthracycline comprises doxorubicin.

Preferably, the GLP-1 receptor agonist and the chemotherapeutic agent are administered by subcutaneous injection.

Preferably, the DPP-4 inhibitor is administered orally (e.g., sitagliptin, vildagliptin, saxagliptin, algoliptin, lingaliptin). In some embodiments, suitable chemotherapeutic agents are administered orally. In some embodiments, where oral administration is not suitable for a desired chemotherapeutic agent, a different mode of administration is used, for example subcutaneous injection.

The chemotherapeutic agent may optionally be provided in a combined dosage form, together with the GLP-1 receptor agonist or the DPP-4 inhibitor. Alternatively, the chemotherapeutic agent may be provided in a separate dosage form, for co-administration or sequential administration, either before or after administration of the GLP-1 receptor agonist or DPP-4 inhibitor.

In embodiments wherein the chemotherapeutic agent is doxorubicin, administration may comprise, for example, subcutaneous injection at intervals of 7 days, or 21-28 days. Administration may therefore be daily, or at intervals of, for example, about 2 days, about 7 days, about 14 day, about 21 days, about 28 days. Preferably, administration is at intervals of 7 days or from about 21 days to about 28 days.

Preferably, dosages of doxorubicin when used together with a GLP-1 receptor agonist such as exendin-4, or with a DPP-4 inhibitor such as sitagliptin, vildagliptin, algoliptin, lingaliptin or saxagliptin in accordance with embodiments of the present invention are in the range of from about 40 to about 60 mg/m² every 21 days or 20 mg/m² every 7 days, thus enhancing its activity. Alternatively, doxorubicin doses can be reduced by about 50%, to about 20 mg/m² every 21 days, or 10 mg/m² every 7 days thus reducing its toxicity.

The present invention further provides a composition comprising pharmaceutically acceptable amounts of a GLP-1 receptor agonist and a chemotherapeutic agent as an additional active pharmaceutical ingredient, respectively selected from any of the GLP-1 receptor agonists and chemotherapeutic agents described hereinabove.

The composition may optionally be provided in extended-release form, as described above with regard to exendin-4 alone.

According to some embodiments, the composition comprises exendin-4 as the GLP-1 receptor agonist, and doxorubicin as the chemotherapeutic agent.

The present invention further provides a composition comprising pharmaceutically acceptable amounts of a DPP-4 inhibitor and a chemotherapeutic agent as an additional active pharmaceutical ingredient, respectively selected from any of the DPP-4 inhibitors and chemotherapeutic agents described hereinabove.

According to some embodiments, the composition comprises sitagliptin, vildagliptin, algoliptin, lingaliptin or saxagliptin as the DPP-4 inhibitor, and doxorubicin as the chemotherapeutic agent.

As discussed in the Examples section below, the activity of doxorubicin, a commonly used chemotherapy for breast cancer on breast cancer cells, has been shown to be enhanced upon co-administration with exendin-4. Hence, the amount of doxorubin used may be reduced to about one half of that used as the sole chemotherapeutic agent, i.e. to about 20 mg/m² every 21 days, or 10 mg/m² every 7 days, thereby reducing the negative side-effects associated with the chemotherapeutic agent at the dosage used when administered as sole active pharmaceutical ingredient.

According to some embodiments of the present invention, there is provided a composition comprising a combination of at least two active pharmaceutical ingredients, at least one of which is a GLP-1 receptor agonist (e.g., exendin-4) or a DPP-4 inhibitor (e.g., sitagliptin, vildagliptin, algoliptin, lingaliptin or saxagliptin), and at least one of which is a chemotherapeutic agent (e.g., doxorubicin), wherein the amount of GLP-1 receptor agonist and amount of the chemotherapeutic agent alone is insufficient to achieve the therapeutic effect achieved by the administration of the combination of two or more of the active pharmaceutical ingredients. The composition of the present invention comprises, in addition to the active pharmaceutical ingredients, a pharmaceutically acceptable carrier, and may optionally further comprise one or more pharmaceutically acceptable excipients.

Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered subcutaneously. Saline solutions and aqueous dextrose and glycerol solutions can also be employed as liquid carriers, particularly for injectable solutions.

If desired, solutions of the above dosage compositions may be thickened with a thickening agent such as methylcellulose. They may be prepared in emulsified form, such as either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton).

In general, the composition of the present invention is prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

Exemplary embodiments of the invention are discussed hereinbelow with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

As noted above, in some embodiments, the teachings of the present invention are directed to treating cancer to reduce or eliminate cancerous tumors and metastatic cells and tumors.

In some embodiments, the teachings of the present invention are implemented to treat cancer as an adjuvant treatment, that is to say together with known modalities of cancer treatment.

In some embodiments, the teachings of the present invention are implemented to treat cancer as a neo-adjuvant treatment, for example to reduce the size of a tumor prior to surgical excision thereof.

In some embodiments, the teachings of the present invention are implemented prophylactically. For example, in some embodiments, the present invention is implemented on a person who has not yet been diagnosed with cancer but is a member of a group at high risk of being diagnosed with cancer, for example has a genetic inclination to cancer (family history), a pathological indication of pre cancer (e.g., pre breast cancer), DCIS (ductal carcinoma in situ), clinically significant alcohol use, age or use of HRT (hormone replacement therapy). For example, in some embodiments, the present invention is implemented on a person whose cancer is in remission (complete or partial) but may be susceptible to a return of the disease.

### Examples

### Materials and Methods

Chemicals, antibodies and constructs: Exendin-4, GLP-1, exendin9-39, didoxyadenosine, AICAR, compound C and w were obtained from Sigma (St. Louis, Mo.). Antibodies used in this study: anti-p53, -PARP, -p21 (Santa Cruz Biotechnology, Santa Cruz, Calif.), anti-phospho-AKT1 (S473), total pan-AKT, (Cell Signaling Technology, Danvers, Mass.), anti-diphosphorylated and -total ERK 1/2 (Sigma). The GLP-1R adenovirus vector was a generous gift of D. Drucker (Toronto).

### Immunofluorescence:

Cells: Breast cancer cell lines were obtained from the American Type Culture Collection (Manassas, Va.).

Real Time Reverse Transcription-PCR (RT-PCR): Total RNA was extracted using the RNA isolation kit (Sigma) and processed to cDNA with RevertAid (Fermentas, Vilnius, Lithuania). Primers were designed using Primer Express (Applied Biosystems, Foster City, Calif.) and synthesized by IDT (Coralville, Iowa). GLP-1R 210-877 specific primers were: Forward 5′-, reverse 5′-. GLP-1R 120-190 primers: Forward 5′-reverse 5′-. Amplification reactions were performed with Platinum qPCR SuperMix (Invitrogen) in triplicates in an ABI Prism™ 7000 (Applied Biosystems) as previously described (Wolf et al., 2007). PCR conditions: 50° C.—2 min, 95° C.—2 min, followed by 40 cycles of: 95° C.—15 sec, 60° C.—45 sec.

Western blot analysis: Cells were harvested and lysed for total protein extraction in RIPA buffer (50 mM Tris-Cl pH 7.4, 150 mM NaCl, 1% NP-40, 0.25% Na-deoxycholate, 1 mM EDTA, 1 mM NaF) together with a protease inhibitor cocktail (Sigma). 50 μg protein extracts were loaded on 10% polyacrylamide gels, separated electrophoretically and blotted from the gel onto nitrocellulose membrane (Schleicher & Schuell Bioscience GmbH, Dassel, Del.). The membranes were then immunoblotted with the indicated antibodies. Band intensities were quantified using ImageJ software.
Colony assays: 200 cells/well were plated on 12-well plate. Twenty-four hrs later medium was replaced and cells were incubated with exendin-4 (5 nM) or control vehicle. Medium was replaced twice a week and at day 14, the cells were stained using crystal violet. Quantification of the results was performed using ImageJ.

MTT viability assay: 3.5×10³ cells/well were plated in 96-well plates, cultured in the appropriate culture media, and treated as indicated, cells were cultured for two hours with 500 μg/ml MIT reagent (Sigma-Aldrich, St. Louis, Mo.). The medium was aspirated, and the cells were dissolved by dimethyl sulfoxide (DMSO). Absorbance of the formazan product was measured by an enzyme-linked immunosorbent assay reader.

Cell cycle assays: For cell cycle assays, 1×10⁶ cells were cultured in the appropriate culture media containing 10% FCS, and treated with either control vehicle or various concentrations of HNK as indicated for 24 h. Following treatment, the cells were harvested, fixed in methanol and stained with propidium iodide (PI, Abcam, Cambridge, Mass.). Flow cytometry was performed at the Flow Cytometry Core facility of Cedars-Sinai Medical Center, using FACScan (Becton Dickinson, Franklin Lakes, N.J.).

Apoptosis analysis: For apoptosis analysis, 1×10⁶ cells were placed in the appropriate culture media containing 10% FCS, and treated with either control vehicle or various concentrations of exendin-4 for 48 hr. Following treatment, cells were harvested, and stained with PI and Annexin V, using the Annexin V-PE Apoptosis Detection Kit I (MBL) according to the manufacturer protocol. Flow cytometry was performed using FACScan (Becton Dickinson, Franklin Lakes, N.J.).

cAMP measurements: For cAMP measurement, a radioimmunoassay was carried out as follows: cells and 100 μl media were snap frozen in liquid nitrogen, thawed and diluted in buffer acetate. Samples were acetylated to increase sensitivity using 1 volume of acetic anhydride and 2 volumes of triethylamine. ¹²⁵I-cAMP and anti cAMP antibody were added to samples and incubated over night at 4° C. Bound cAMP was precipitated and counted in a γ-counter, and concentration determined using a standard curve and positive controls.

Animal studies: All animals experiments were performed under institutional guidelines established for the Animal Core Facility at the Sheba Medical Center. MDA-MB-231 cells were harvested, washed twice with sterile PBS, counted and re-suspended in Matrigel (BD Biosciences, San Jose, Calif.). Six-week-old female athymic nude mice were injected subcutaneously in both flanks with cells at a density of 1×10⁶ viable cells/100 μl. Five days later the mice were implanted subcutaneously with 28-day osmotic pumps (Alzet) delivering vehicle (PBS) or exendin-4 continuously (500 ng/day, 2 μg/day). Five mice were used in each group. Tumor size was measured with a linear caliper for up 4 weeks, and the volume was estimated by using the equation V=(a×b²)×0.5236, where “a” is the larger dimension and “b” the perpendicular diameter.

Statistical analysis: The study variables were compared between the study groups using Fisher's exact test for categorical variables.

### Effect of Exendin-4 on Cell Viability

The effect of various concentrations of exendin-4 on the viability of breast cancer cells was studied, using two breast cancer cell lines: the hormone-positive breast cancer cell line, MCF-7, and the hormone-negative breast cancer breast cancer cell line, MDA-MB-231. Human embryonic kidney HEK-293 and primary liver cells were used as controls.

3.5×10³ cells/well were plated in 96-well plates, cultured in the appropriate culture media containing 10% fetal calf serum, and treated with either control vehicle (phosphate buffered saline (PBS)), or various concentrations of exendin-4 (Sigma-Aldrich, St. Louis, Mo.), as indicated. Medium was replaced once after 48 hrs.

After 24, 48 and 72 hrs of incubation at 37° C., 5% CO2, the cells were cultured for four hours with 10% 3-(4,5-Dimethylthiazol-2-yl)-2,5-diphenyltetrazolium bromide (MTT) reagent (5 mg/ml; Sigma-Aldrich). The medium was aspirated, and the cells were dissolved by dimethyl sulfoxide (DMSO). Absorbance of the formazan product was measured by an enzyme-linked immunosorbent assay reader.

### Effect of Exendin-4 on Cell Growth

The present inventors could not detect significant levels of the GLP-1 receptor in breast cancer cells by conventional PCR or real-time PCR (data not shown), hence it was decided to evaluate the effect of exendin-4 on breast cancer cell growth.

The effect of 5 nM exendin-4 on growth of MCF-7, MDA-MB-231, and primary liver cells was assessed by colony assays. Cells were plated at low density (200 cells/well in 12-well plate), cultured in the appropriate culture medium containing 10% fetal calf serum, with or without 5 nM exendin-4. Medium was replaced every 48 hours. After 10 days medium was aspirated, cells were washed twice with PBS, fixated with 70% methanol for 5 min, and stained with crystal violet for 5 min and washed with distilled water.

### Effect of Exendin-4 on Expression of p53 and p21

The effect of exendin-4 (5 nM) on expression of p53 and p21 in MCF-7 and MDA-MB-231 breast cancer cells and hepatic cells was studied, using Western blotting.

10⁶ MCF-7, MDA-MB-231 or human primary liver cells were plated in 6 cm dishes in growth medium, supplemented with 10% FCS. Twenty-four hrs later, medium was removed and fresh medium (with serum), containing either 5 nM exendin-4 or PBS, was added to the cells. After a further 24 hrs cells were washed twice with ice-cold. PBS and frozen in liquid nitrogen.

Cells were harvested and lysed for total protein extraction in RIPA buffer (50 mM Tris-Cl pH 7.4, 150 mM NaCl, 1% NP-40, 0.25% Na-deoxycholate, 1 mM EDTA, 1 mM NaF) together with a protease inhibitor cocktail (Sigma). 50 μg protein extracts were loaded on 10% polyacrylamide gels, separated electrophoretically and blotted from the gel onto nitrocellulose membrane (Schleicher & Schuell Bioscience GmbH, Dassel, Del.). The membranes were then immunoblotted with the indicated antibodies (p53 1:1000 dilution; p21 1:500 dilution and PARP 1:1000 dilution, all antibodies from Santa Cruz, Calif.).

### Effect of Exendin-4 on Camp Levels in MCF-7 Cells

1×10⁴ cells were plated on 12-well dish, and treated the next day for 1, 5, 15, 30 and 60 minutes with 5 nM exendin-4, or control vehicle at indicated times. The effect of various concentrations of exendin-4 was also studied.

The effects of exendin-4 (5 nM), GLP-1 (5 nM), forskolin (1 μM), exendin-4 inhibitor exendin (9-39) (100 nM), adenlyate cyclase inhibitor 2′5′-dideoxy adenosine (30 μM), and combinations thereof were studied. The cells were treated for 5 minutes with the different substances, except for 2′5′-dideoxy adenosine, where cells were preincubated for 60 minutes before either the assay or addition of exendin-4.

Effect of Exendin-4 in Combination with Doxorubicin on Viability of MCF-7 Cells

Doxorubicin is a commonly used chemotherapy against breast cancer. The ability of exendin-4 to enhance the cytotoxic activities of doxorubicin was tested using MIT assay on MCF-7 cells.

2.5×10³ MCF-7 cells/well were plated in 96-well plates, cultured in the appropriate culture media containing 10% fetal calf serum, and either treated with either control vehicle (phosphate buffered saline (PBS)), 2.5 nM exendin-4, or 50 nM doxorubicine (Sigma), or co-treated with doxorubicine and exendin-4. Media and treatments were changed once after 48 hrs. After 72 hrs of incubation at 37° C., 5% CO2, the cells were cultured for four hours with 10% MIT reagent (5 mg/ml; Sigma-Aldrich). The medium was aspirated, and the cells were dissolved by dimethyl sulfoxide (DMSO). Absorbance of the formazan product was measured by an enzyme-linked immunosorbent assay reader.

### Effect of Exendin-4 on Activation of CREB and p38

In the pancreas GLP-1/exendin-4 has been found to activate a subset of pathways, including cAMP, CREB, AKT and ERK. In order to characterize the signaling pathways activated by exendin-4 and GLP-1 in breast cancer cells, MCF-7 cells were treated with exendin-4 or GLP-1.

### In Vivo Effect of Exendin-4 in Mice

In order to evaluate the effect of exendin-4 on inhibition of tumor growth of breast cancer cells in vivo, MDA-MB-231 cells were injected into both flanks of 6-week old athymic female mice (1×106 cells per injection, 5 mice per group, 2 tumors per mouse), and tumor growth was monitored weekly. These cells were chosen based on their ability to easily form tumors in nude mice and their sensitivity to exendin-4. The mice were implanted subcutaneously with 28-day osmotic pumps (Alzet0 delivering vehicle (PBS) or exendin-4 continuously (500 ng/day, 2 μg/day).

### Results and Discussion

### Influence of Exendin-4 on Cell Viability

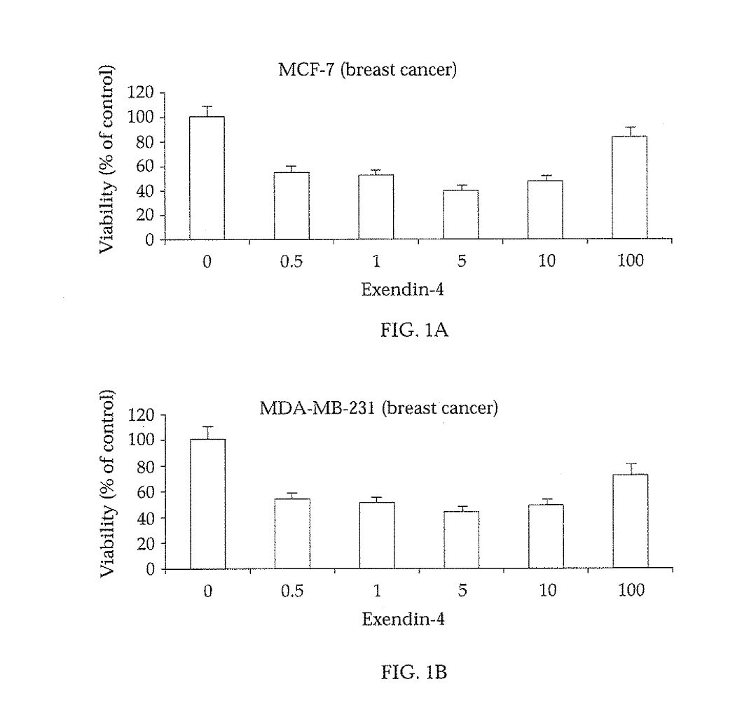

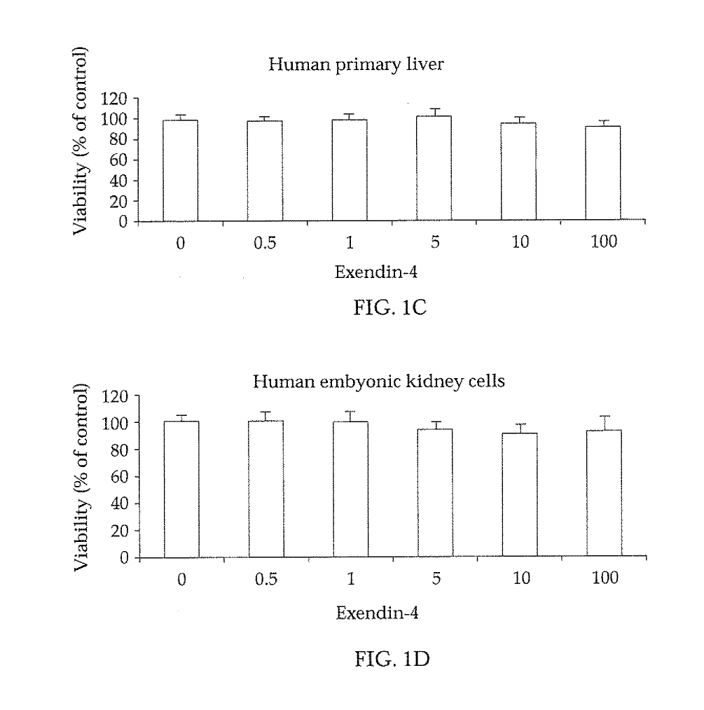

As shown in FIGS. 1A and 1B, MTT assays of MCF-7 and MDA-MB-231 cells, respectively, conducted at 48 hours, revealed that exendin-4 reduced cell viability in a dose-dependent manner, achieving maximal effect of 40% reduction at a dosage of 5 nM. Exendin-4 did not affect growth of HB2 (not shown), HEK293, primary liver cells (FIG. 1C) or human embryonic kidney cells (FIG. 1D). Doses of 1-10 nM were shown to be effective, with higher doses showing reduced effect.

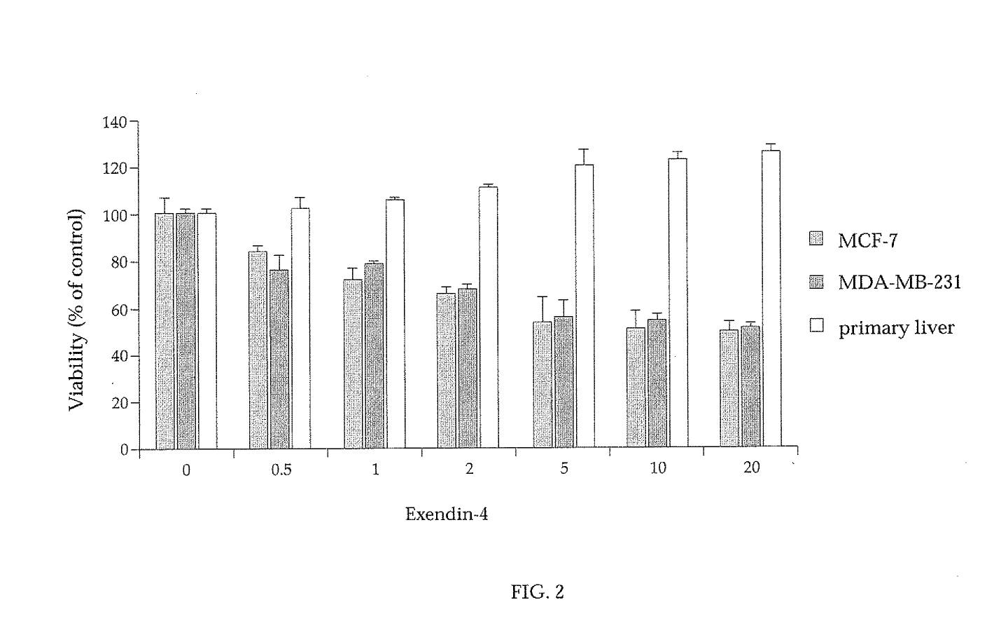

The effect of exendin-4 was substantially the same on both breast cancer cell lines, indicting that the effect of exendin-4 does not require the presence of hormone receptors. The results are summarized in FIG. 2.

### Effect of Exendin-4 on Cell Growth

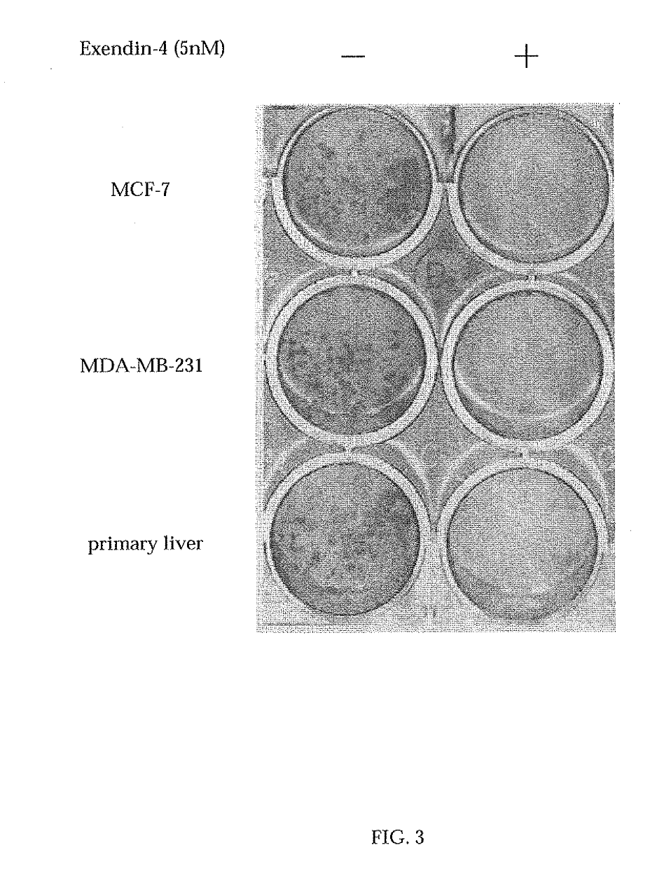

As shown in FIG. 3, after 10 days, exendin-4 significantly reduced the number of colonies formed by MCF-7 cells and MDA-MB-231 cells but did not affect colony formation of the primary liver cells.

Colony assay was conducted to MCF-7 cells infected with GLP-1R-expressing adenovirus or GFP-expressing adenovirus, serving as control. Exendin-4 reduced colony number of GFP-infected cells (FIG. 1B). Interestingly, expression of GLP-1R in MCF-7 resulted in an increase of colony number and size; however, treating the GLP-1R-expressing MCF-7 with exendin-4 decreased colony number (FIG. 1B).

### Effect of Exendin-4 on Expression of p53 and p21 and Apoptosis

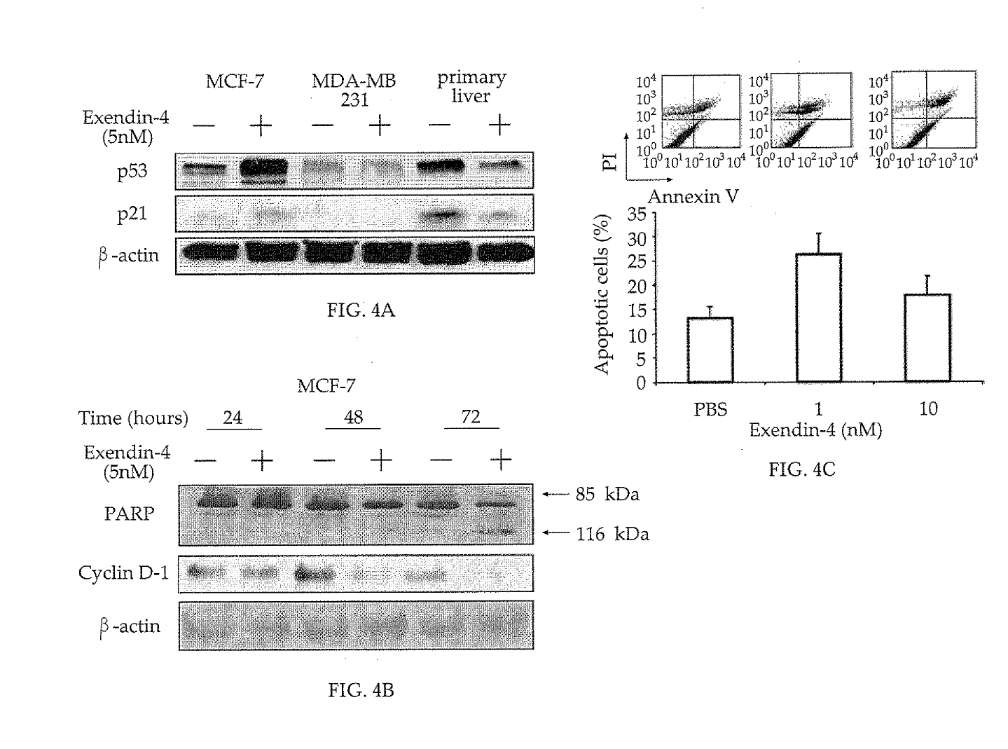

As shown in FIGS. 4A to 4C, exendin-4 was found to increase expression of p53 and p21 proteins, both of which are associated with cell cycle arrest in MCF-7 and MDA-MB-231 cells, but to cause down-regulation in liver primary cultures. MDA-MB-231 cells are p53 mutated, therefore no difference in p53 can be detected. Since p21 expression is regulated by several factors, and p53 is an important p21 regulator, p21 is not affected in MDA-MB-231 cells.

In order to ascertain that different p21, p53 and PARP expression levels are due to a true cellular effect, and not due to unequal loading of samples, levels of β-actin, which is a ‘housekeeping’ gene, were measured and its expression was found to remain constant under different treatments.

p53 is a transcription factor that regulates the cell cycle and hence functions as a tumor suppressor. p53 can activate DNA repair proteins when DNA damage has occurred, and can hold the cell cycle at the G₁/S regulation point such that the DNA repair proteins have sufficient time to repair the damage, enabling the cell to complete the cell cycle. p53 can also initiate apoptosis if DNA damage proves to be irreparable.

p21 is a human gene on chromosome 6, that encodes a cyclin-dependent kinase inhibitor that directly inhibits the activity of cyclin-DKK2 and cyclin-CDK4 complexes, which are essential for G₁/S phase cell cycle transition. p21 thus functions as a regulator of cell cycle progression at the S phase. The expression of p21 is controlled by p53.

Inhibition of p53 and p21 therefore indicates a role of exendin-4 in tumor suppression.

The effect of exendin-4 on apoptosis was assayed with annexin-V staining, and it was found that exendin-4 induces apoptosis in MCF-7 and MDA-MB-231 cells (FIG. 4B). No effect on cell cycle was noted (data not shown).

As shown in FIG. 4C, exendin-4 increases expression of the cell cycle protein, cyclin D1 and induces apoptosis as indicated by PARP cleavage in MCF-7 cells after 48 and 72 hr.

PARP, a 116 KDa nuclear poly (ADP-ribose) polymerase, appears to be involved in DNA repair in response to environmental stress. The protein can be cleaved by caspases, including caspase-3 in vivo. In human PARP, the cleavage occurs between Asp214 and Gly215, which separates the PARP amino-terminal DNA binding domain (24 Da) from the carboxy-terminal catalytic domain (89 Da). PARP helps cells to maintain their viability; cleavage of PARP facilitates cellular disassembly and serves as a marker of cells undergoing apoptosis. Therefore, increase in PARP cleavage product, as seen in MCF-7 cells treated with exendin-4, indicates that the cells undergo apoptosis.

### Effect of Exendin-4 on Camp Levels in MCF-7 Cells

cAMP is a second messenger, used for intracellular signal transduction, such as transferring the effects of hormones, including glucagon, which cannot cross the cell membrane. Binding of hormone to adenylate cyclase stimulatory G-coupled receptors causes activation of adenylate cyclase, which is located at the cell membrane, which in turn leads to synthesis of cAMP from adenosine triphosphate (ATP). Upon receptor activation, cAMP levels peak rapidly (within less than about a minute), and the increase in cAMP last about 15 minutes.

Exendin-4 has been shown to increase cAMP levels in pancreatic cells. The effects of exendin-4 on cAMP levels in MCF-7 cells were studied using radioimmunoassay (RIA).

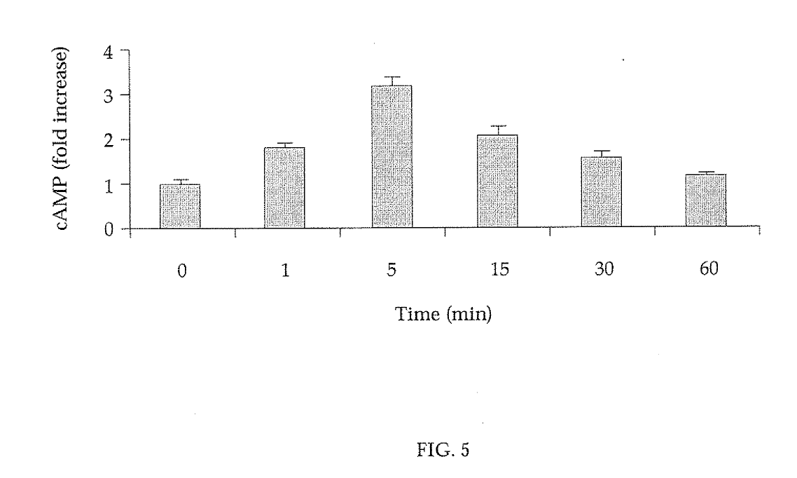

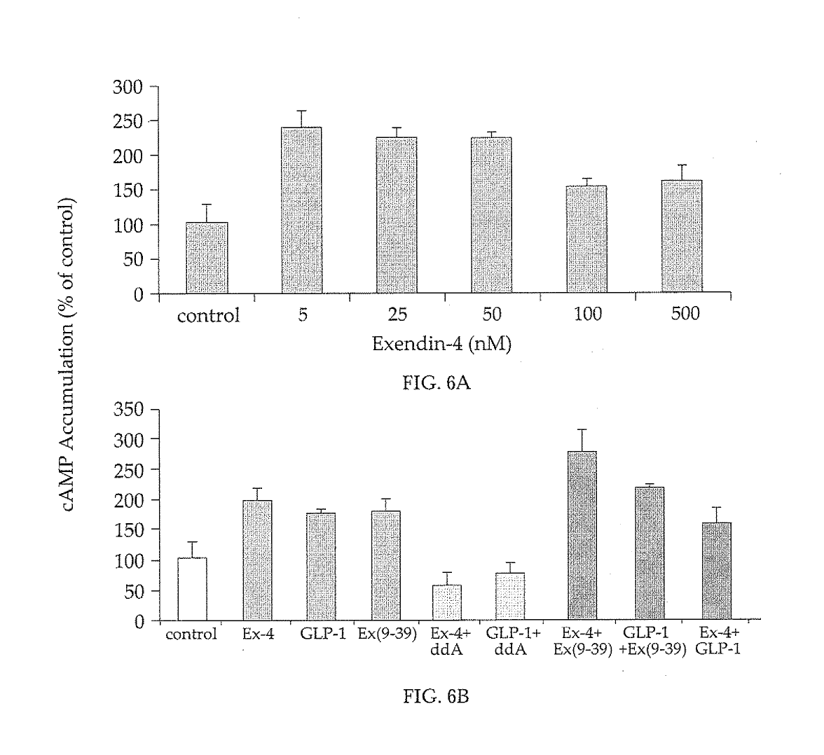

As shown in FIGS. 5 and 6A, cAMP levels in MCF-7 cells were increased following exendin-4 treatment, with maximal effect of 2.5-fold at 5 minutes and at a concentration of 5 nM.

The effect of exendin-4 on cAMP levels were compared to the effects of GLP-1, the GLP1R antagonist exendin (9-39), and the adenylate cyclase inhibitor 2′5′ dideoxy-adenosine (ddA), (FIG. 6B). The effects of GLP-1 and exendin-4 on cAMP accumulation were similar. Interestingly, exendin(9-39) increased cAMP accumulation similarly to exendin-4 and GLP-1. Interestingly, a combination of either exendin-4 with GLP-1 or exendin(9-39) further elevated cAMP production (FIG. 6B), suggesting a synergistic effect.

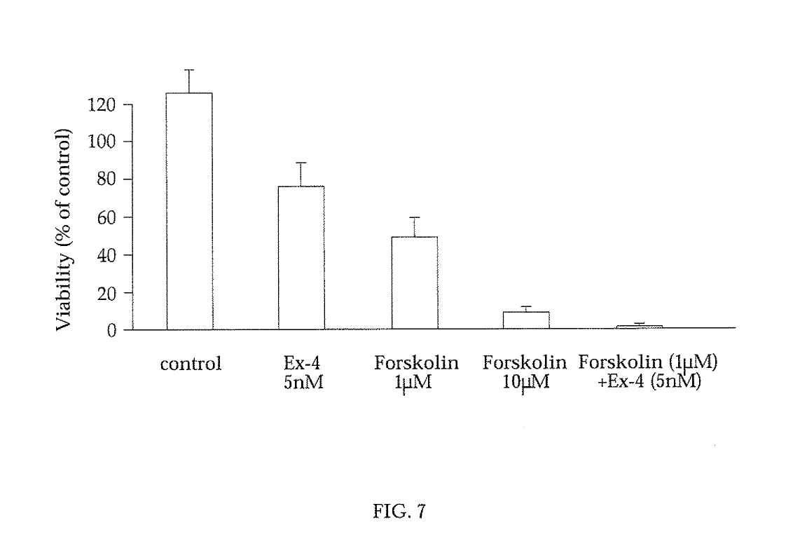

The growth inhibitory effect of exendin-4 was compared to that of the cAMP inducer forskolin and a significant growth inhibitory effect of forskolin on MCF-7 cells was seen (FIG. 7). Forskolin inhibited cell viability by 60% at 1 μM and no viable cells were detected when treated with forskolin (1 μM) and exendin-4 (5 nM).

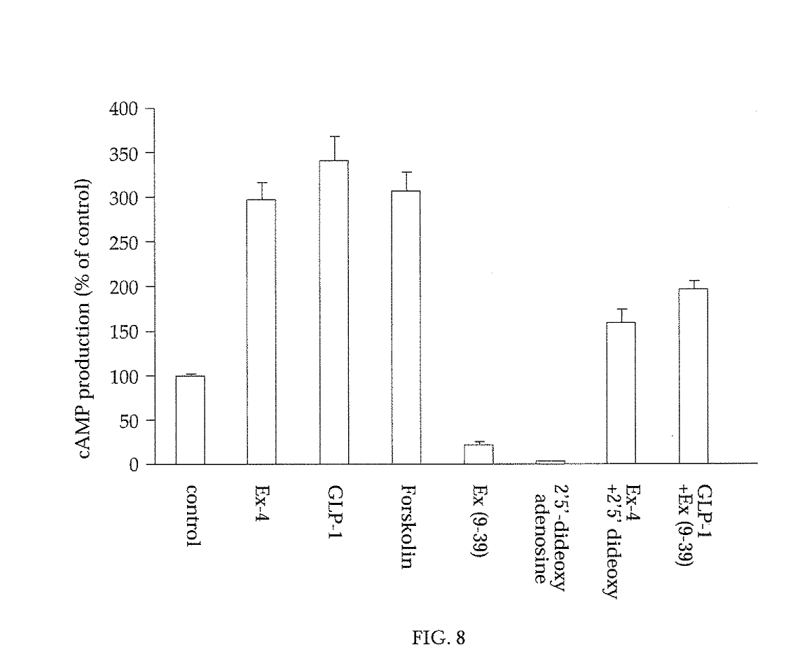

Forskolin elevated cAMP levels 6-fold, whereas cAMP production was inhibited following treatment with ddA (FIG. 8).

Hence, increased cAMP levels suggest the involvement of the G-coupled protein receptor in mediating the response of cells to exendin-4.

Effect of Exendin-4 in Combination with Doxorubicin on Viability of MCF-7 Cells

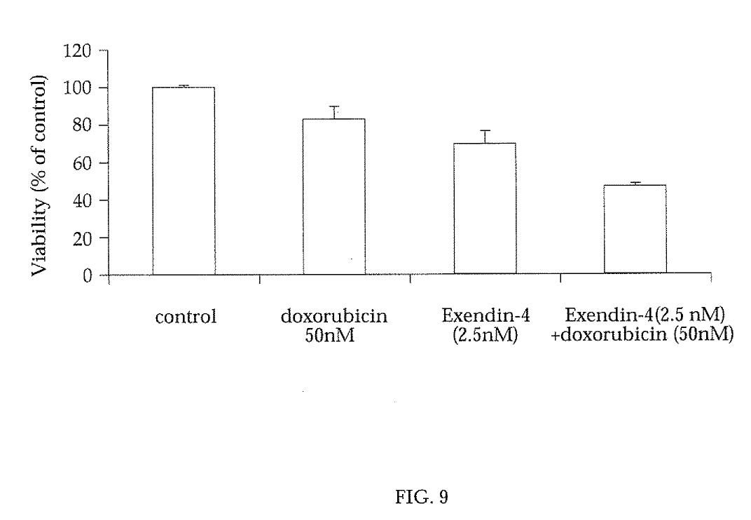

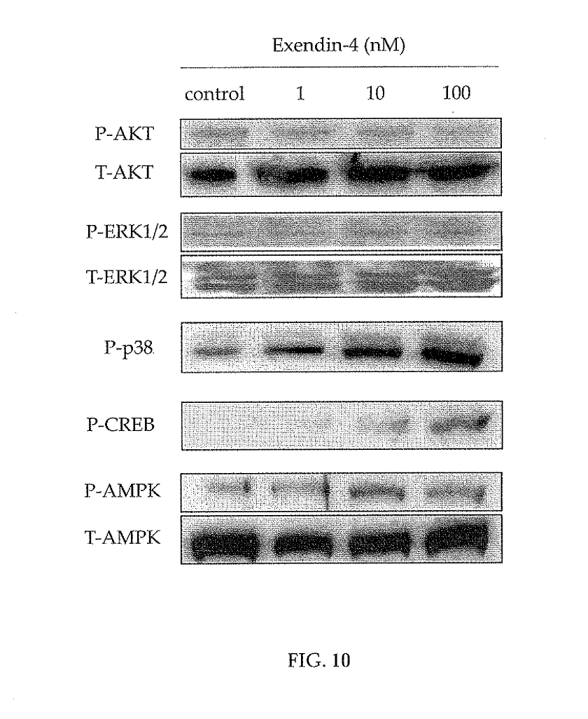

As shown in FIG. 9, the effect of exendin-4 in combination with doxorubicin on reduction of MCF-7 breast cancer cell line viability after 72 hours was greater than that of either agent alone. While doxorubicin reduced viability by 20% and exendin-4 by 30%, the combination reduced growth by 50%.

### Effect of Exendin-4 on Activation of CREB and p38

In pancreatic cells, exendin-4 signals through AKT and ERK, among other signaling pathways. MCF-7 cells treated with either exendin-4 or GLP-1 for 15 minutes resulted in CREB phosphorylation, but, neither ERK1/2 nor AKT were affected by them (FIG. 100. On the other hand, the stress-activated MAPK p38 was activated by exendin-4 and GLP-1. Interestingly, exendin(9-39) by itself, or with exendin-4 enhanced p38 and CREB phosphorylation.

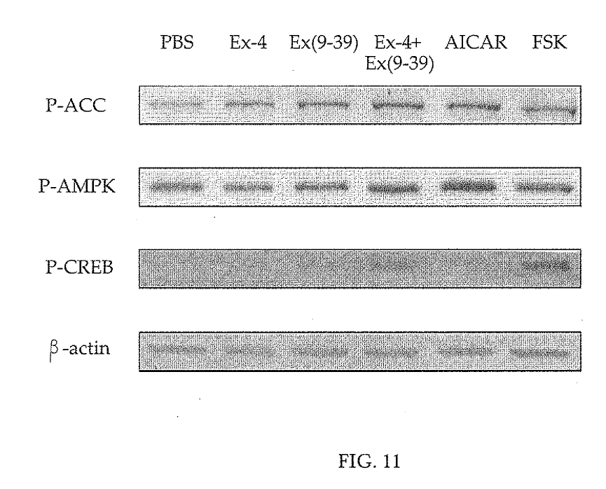

AMP kinase (AMPK) is a stress kinase whose activation depends on energy balance in the cells. GLP-1 activates AMP kinase in the heart and brain, and its activation was also found to inhibit breast cancer growth. The present inventors studied AMPK activation in MCF-7 cells and found that AMPK and its downstream target ACC are phosphorylated following exendin-4, GLP-1 and exendin(9-39) treatment, comparable to the AMPK activator AICAR (FIG. 11). It was also shown that co-treatment with exendin-4 and exendin9-39 further induced CREB phosphorylation.

### In Vivo Effect of Exendin-4

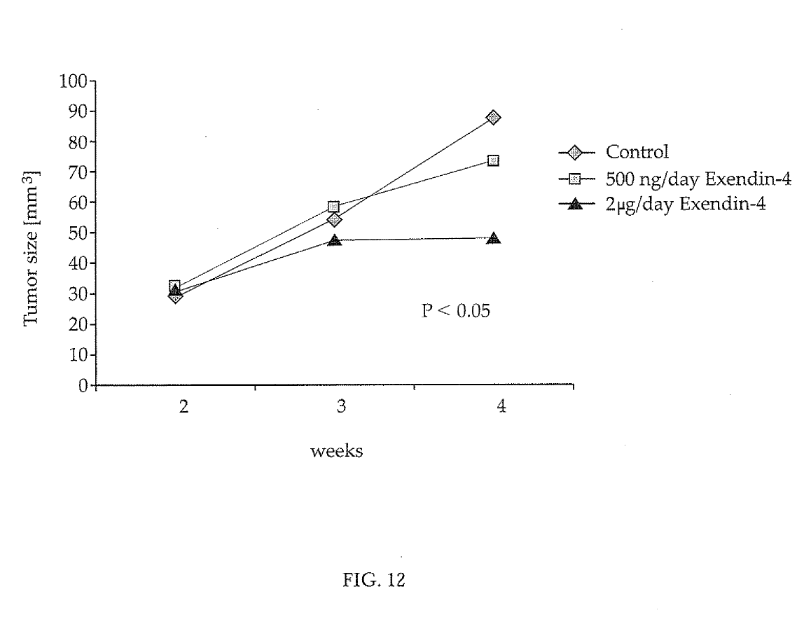

As shown in FIG. 12, treatment of nude mice in vivo with 2μ exendin-4 resulted in a significant dose-response attenuation of tumor growth, with a reduction in tumor size of approximately 50% at 4 week in treated mice, as compared to control mice. 500 ng/day exendin-4 resulted in a reduction of tumor size of approximately 20% at 4 weeks, as compared to control.

These results clearly demonstrate that exendin-4 is highly effective in the treatment of breast cancer.

### GLP-1 Receptor Agonists for Treatment of Cancer

### Breast Cancer

Fifty-day-old female obese Zucker rats (highly susceptible to developing obesity related-cancer) are orally gavaged with 65 mg/kg DMBA (7,12-dimethylbenz(a)anthracene).

All the rats are thereafter fed an obesity-maintaining diet (rich in lard and oils). 50 rats are given a placebo, another 100 rats are also given daily subcutaneous doses of GLP-1 receptor agonists: exenatide (n=50, Byetta®) and liraglutide (n=50, Victoza®) and another 100 rats are also given daily subcutaneous doses of GLP-1 receptor agonists: exenatide (n=50, Byetta®) and liraglutide (n=50, Victoza®) together with intravenous doses of Doxil® (a commercial doxorubicin composition by Johnson and Johnson, New Brunswick, N.J., USA).

The rats are sacrificed 100 days after DMBA treatment and examined for mammary tumors. A significantly greater incidence of tumors is observed in the fifty normally fed rats compared to the 200 rats which also receive the GLP-1 receptor agonists.

### Colon Cancer

Azoxymethane is subcutaneously administered to obese rats to initiate the development of colon cancer, as described in Weber RV in Dig Dis Sci 2000, 45, 890-895.

All the rats are thereafter fed an obesity-maintaining diet (rich in lard and oils). 50 rats are given a placebo, another 100 rats are also given daily subcutaneous doses of GLP-1 receptor agonists: exenatide (n=50, Byetta®) and liraglutide (n=50, Victoza®) and another 100 rats are also given daily subcutaneous doses of GLP-1 receptor agonists: exenatide (n=50, Byetta®) and liraglutide (n=50, Victoza®) together with intravenous doses of Doxil® (a commercial doxorubicin composition by Johnson and Johnson, New Brunswick, N.J., USA).

The rats are sacrificed 100 days after azoxymethane treatment and examined for tumors in the colon. A significantly greater incidence of tumors is observed in the fifty normally fed rats compared to the 200 rats which also receive the GLP-1 receptor agonists.

### DPP-4 Inhibitors for Treatment of Cancer

### Breast Cancer

Fifty-day-old female obese Zucker rats (highly susceptible to developing obesity related-cancer) are orally gavaged with 65 mg/kg DMBA (7,12-dimethylbenz(a)anthracene).

All the rats are thereafter fed an obesity-maintaining diet (rich in lard and oils). 50 rats are given a placebo while another 250 rats are also given daily oral 2 mg/kg doses of DPP-4 inhibitors: sitagliptin (n=50), vildagliptin (n=50), saxagliptin (n=50), algoliptin (n=50) and linagliptin (n=50) and another 250 rats are also given daily oral 2 mg/kg doses of DPP-4 inhibitors: sitagliptin (n=50), vildagliptin (n=50), saxagliptin (n=50), algoliptin (n=50) and linagliptin (n=50) together with intravenous administration of Doxil® (a commercial doxorubicin composition by Johnson and Johnson, New Brunswick, N.J., USA).

The rats are sacrificed 100 days after DMBA treatment and examined for mammary tumors. A significantly greater incidence of tumors is observed in the fifty normally fed rats compared to the 500 rats which also receive the DPP-4 inhibitors.

### Colon Cancer

Azoxymethane is subcutaneously administered to obese rats to initiate the development of colon cancer, as described in Weber R V in Dig Dis Sci 2000, 45, 890-895.

All the rats are thereafter fed an obesity-maintaining diet (rich in lard and oils). 50 rats are given a placebo while another 250 rats are also given daily oral 2 mg/kg doses of DPP-4 inhibitors: sitagliptin (n=50), vildagliptin (n=50), saxagliptin (n=50), algoliptin (n=50) and linagliptin (n=50) and another 250 rats are also given daily oral 2 mg/kg doses of DPP-4 inhibitors: sitagliptin (n=50), vildagliptin (n=50), saxagliptin (n=50), algoliptin (n=50) and linagliptin (n=50) together with intravenous administration of Doxil® (a commercial doxorubicin composition by Johnson and Johnson, New Brunswick, N.J., USA).

The rats are sacrificed 100 days after azoxymethane treatment and examined for tumors in the colon. A significantly greater incidence of tumors is observed in the fifty normally fed rats compared to the 500 rats which also receive the DPP-4 inhibitors.

These results demonstrate the claimed hereinbelow.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

To the extent that section headings are used, they should not be construed as necessarily limiting.

1-50. (canceled)

51. A method of treating cancer comprising increasing the concentration of a glucagon-like peptide 1 (GLP-1) receptor agonist in the body of a subject in need thereof by at least one administration selected from the group consisting of:
administering to said subject an effective amount of a composition comprising a pharmaceutically effective amount of a glucagon-like peptide 1 (GLP-1) receptor agonist; and
administering to said subject an effective amount of a composition comprising a pharmaceutically effective amount of a DPP-4 inhibitor, without administering a DPP8/9 inhibitor.

52. The method of claim 51, wherein said cancer is a cancer related to obesity and diabetes.

53. The method of claim 52, wherein said cancer is breast cancer.

54. The method of claim 51, wherein said cancer is metastatic cancer.

55. The method of claim 54, wherein a metastasis is present in a site selected from the group consisting of the lymph nodes, bones, liver, lungs, and brain

56. The method of claim 55, wherein said metastasis is present in the brain.

57. The method of claim 56, wherein said metastatic cancer is metastatic breast cancer.

58. The method of claim 53, wherein said breast cancer is hormone positive

59. The method of claim 53, wherein said breast cancer is hormone negative.

60. The method of claim 51, wherein said GLP-1 receptor agonist is a GLP-1 derivative, analog, mimetic or salt.

61. The method of claim 60, wherein said GLP-1 receptor agonist comprises exendin-4 (SEQ ID NO: 1), or an analog, mimetic, derivative or salt thereof.

62. The method of claim 51, wherein said DPP-4 inhibitor is selected from the group consisting of sitagliptin, vildagliptin, saxagliptin, algoliptin, lingaliptin and Val-Pyr, derivatives thereof, analogs thereof, mimetics thereof, and salts thereof.

63. The method of claim 51, wherein said composition further comprises at least one chemotherapeutic agent.

64. The method of claim 63, wherein said at least one chemotherapeutic agent comprises an agent selected from the group consisting of an alkylating agent, an antimetabolite, an anthracycline, a plant alkaloid, a topoisomerase inhibitor, a hormone receptor modulator, a hormone level modulator, or combinations thereof.

65. The method of claim 64, wherein said anthracycline is selected from the group consisting of daunorubicin, doxorubicin, epirubicin, idarubicin, mitoxantrone and valrubicin, and combinations thereof.

66. The method according to claim 65, wherein said anthracycline comprises doxorubicin.

67. A method of treating cancer comprising administering to a subject in need thereof a pharmaceutically effective amount of a composition comprising exendin-4 (SEQ ID NO: 1) or an analog, mimetic, derivative or salt thereof.

68. A composition comprising pharmaceutically effective amounts of exendin-4 and doxorubicin, and a pharmaceutically effective carrier.

69. A method of treating breast cancer comprising administering to a subject in need thereof an effective amount of a composition comprising a pharmaceutically effective amount of a DPP-4 inhibitor.


SEQUENCE LISTING (1 sequence)

SEQ ID NO: 1 - protein, 39 aa, Gila Monster
HGEGTFTSDLSKQMEEEAVRLFIEWLKNGGPSSGAPPPS